(19) United States
(12) Patent Application Publication
(10) Pub. No.: US 2026/0179722 A1
(43) Pub. Date: Jun. 25, 2026

(54) METHOD FOR CONSTRUCTING POLYPEPTIDE MOLECULE, AND ELECTRONIC DEVICE

(71) Applicant: Beijing Youzhuju Network Technology Co., Ltd., Beijing (CN)

(72) Inventors: Danqing Wang, Beijing (CN); Zeyu Wen, Beijing (CN); Lei Li, Beijing (CN); Hao Zhou, Beijing (CN)

(21) Appl. No.: 18/714,485

(22) PCT Filed: Nov. 21, 2022
(86) PCT No.: PCT/CN2022/133259

§ 371 Date: May 29, 2024

(30) Foreign Application Priority Data
Dec. 3, 2021 (CN) 202111467002.8

(51) Int. Cl. G16B 30/00; C07K 7/08; G06N 3/045; H04N 19/61; H04N 19/91

(52) U.S. Cl. CPC G16B 30/00; C07K 7/08; G06N 3/045; H04N 19/61; H04N 19/91

(57) ABSTRACT

Embodiments of the present disclosure provide a method for constructing a polypeptide molecule, an apparatus, a device, a storage medium, and a program product. The method described herein comprises: obtaining a set of coding tables of a generation model, the set of coding tables comprising a plurality of discrete coded representations, the generation model comprising a first decoder and a second decoder, the set of coding tables being used for constructing a first input to the first decoder and a second input to the second decoder, the first decoder being used for determining a secondary structure of a polypeptide molecule based on the first input. According to the embodiments of the present disclosure, the polypeptide molecule having higher antibacterial activity can be obtained by considering the secondary structure in the process of constructing the polypeptide molecule.

### CROSS REFERENCE

This application claims the benefit of priority of Chinese invention patent application No. 202111467002.8, filed on Dec. 3, 2021, entitled “Method for Constructing Polypeptide Molecule, and Electronic Device”, which is incorporated herein by reference in its entirety.

### FIELD

Implementations of the present disclosure relate to the field of computers, and more particularly, to a method for constructing polypeptide molecule, an apparatus, a device and a computer storage medium.

### BACKGROUND

Peptides are compounds formed by amino acids linked together by peptide bonds. The Antimicrobial Peptides (AMPs) has shown good effects in broad-spectrum antibiotics and anti-infective treatment. The AMP is an emerging therapeutic agent, which is defined as a short protein of less than 50 amino acids with potent antibacterial activity.

Unlike traditional medicines, the AMP can attach to bacterial membranes and form pores in the bacterial membranes, thereby killing the bacteria. Such a way of destroying bacteria physically is called a “barrel stave”. In such a sterilization process, the antibacterial activity of the AMP is closely related to the secondary structure of the peptide.

### SUMMARY

In a first aspect of the present disclosure, a method for constructing a polypeptide molecule. The method comprises: obtaining a set of coding tables of a generation model, the set of coding tables comprising a plurality of discrete coded representations, the generation model comprising a first decoder and a second decoder, the set of coding tables being used for constructing a first input to the first decoder and a second input to the second decoder, the first decoder being used for determining a secondary structure of a polypeptide molecule based on the first input, and the second decoder being used for determining an amino acid sequence of the polypeptide molecule based on the second input; constructing a first feature representation and a second feature representation based on the plurality of discrete coded representations in the set of coding tables; determining, with the first decoder, a target secondary structure of a target polypeptide molecule according to the first feature representation; and determining, with the second decoder, a target amino acid sequence of the target polypeptide molecule according to the second feature representation.

In the second aspect of present disclosure, an electronic device is provided, including: a memory and a processor; wherein the memory is used to store one or more computer instructions, wherein the one or more computer instructions are executed by the processor to implement the method of the first aspect of this disclosure.

In the third aspect of present disclosure, a computer-readable storage medium is provided, the computer-readable storage medium having one or more computer instructions stored thereon, wherein the one or more computer instructions are executed by a processor to implement the method of the first aspect of this disclosure.

In the fourth aspect of present disclosure, a computer program product is provided, comprising one or more computer instructions, wherein the one or more computer instructions are executed by a processor to implement the method of the first aspect of this disclosure.

Based on this approach, the embodiments of the present disclosure are able to consider the secondary structure in the process of constructing polypeptide molecules, thereby obtaining polypeptide molecules with higher antibacterial activity.

### BRIEF DESCRIPTION OF THE DRAWINGS

The above and other features, advantages and aspects of various embodiments of the present disclosure will become more apparent with reference to the following detailed description taken in conjunction with the accompanying drawings. In the drawings, the same or similar reference numbers represent the same or similar elements, wherein:

FIG. 1A and FIG. 1B show a comparison of the applications of polypeptide molecules with different structures;

FIG. 2 shows a schematic block diagram of a computing device capable of implementing some embodiments of the present disclosure;

FIG. 3 shows a schematic diagram of training a generation model according to some embodiments of the present disclosure;

FIG. 4 shows a schematic diagram of constructing polypeptide molecules with the generation models according to some embodiments of the present disclosure; and

FIG. 5 shows a flow diagram of an example method for constructing polypeptide molecule according to some embodiments of the present disclosure.

### DETAILED DESCRIPTION

Embodiments of the present disclosure will be described in more detail below with reference to the accompanying drawings. Although some embodiments of the disclosure are shown in the drawings, portion disclosure may be implemented in various forms and should not be construed as limited to the embodiments set forth herein. These embodiments are provided for thoroughly and fully understanding of this disclosure. It should be understood that the drawings and embodiments of the present disclosure are for illustrative purposes only and are not intended to limit the scope of the present disclosure.

In the description of embodiments of the present disclosure, the term “include” and similar expressions shall be understood as an open-ended inclusion, that is, “including but not limited to”. The term “based on” should be understood as “based at least in part on”. The term “an embodiment” or “the embodiment” shall be understood as “at least one embodiment”. The terms “first”, “second”, etc. may refer to different or the same object. Other explicit and implicit definitions may be included below.

As discussed above, the antimicrobial peptide AMP, as an emerging therapeutic medicine, has shown good effects in broad-spectrum antibiotics and anti-infective treatment. Specifically, the AMP can physically kill bacteria by destroying bacterial membranes through a “barrel stave” mechanism.

Since most bacterial surfaces are anionic, positively charged amino acids are more likely to bind to the bacterial membrane, while highly hydrophobic amino acids tend to migrate from the solution environment to the bacterial membrane. However, the mechanism of function of the AMP requires not only a reasonable sequence but also an appropriate structure. For example, by forming a helical structure, the AMP can collect hydrophobic amino acids on one side and hydrophilic amino acids on the other side. This ability, called amphipathy, helps antimicrobial peptides to insert into membranes and maintain stable pores with other peptide molecules in the membrane, thereby killing bacteria more effectively.

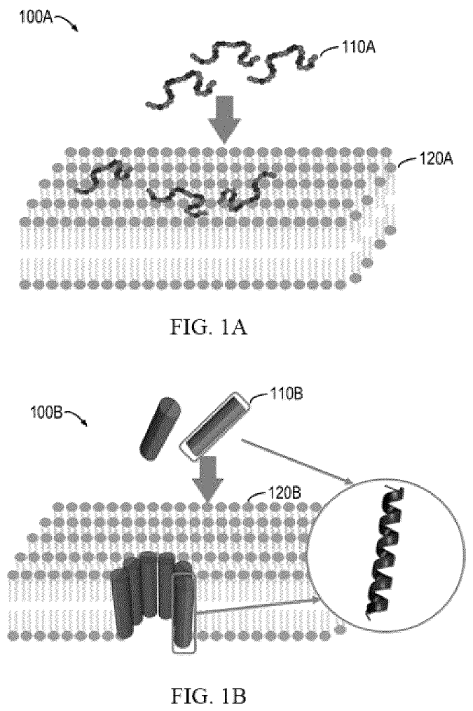

FIG. 1A and FIG. 1B show a comparative schematic diagram of the application of polypeptide molecules with different structures. It can be seen that, as shown in FIG. 1A, the polypeptide molecule 110A can only be attached to the bacterial membrane 120A, but it is difficult to form pores. On the contrary, as shown in FIG. 1B, the polypeptide molecule 110B with a helical structure can more easily form stable pores in the bacterial membrane 120B due to its amphipathy. It can be seen that the secondary structure of the polypeptide molecule will directly affect the antibacterial activity of the polypeptide molecule.

In accordance with implementation of the present disclosure, a scheme for constructing polypeptide molecules is provided. In this scheme, a set of coding tables of the generation model can be obtained, wherein the set of coding tables includes multiple discrete coded representations, the generation model comprising a first decoder and a second decoder, the set of coding tables being used for constructing a first input to the first decoder and a second input to the second decoder, the first decoder being used for determining a secondary structure of a polypeptide molecule based on the first input, and the second decoder is used for determining an amino acid sequence of the polypeptide molecule based on the second input. As an example, the generation model may be a VQ-VAE model (Vector Quantization-Variational Auto encoder).

Further, a first feature representation and a second feature representation may be constructed based on the plurality of discrete coded representations in the set of coding tables. With the first decoder, a target secondary structure of a target polypeptide molecule may be determined according to the first feature representation; and with the second decoder, a target amino acid sequence of the target polypeptide molecule may be determined according to the second feature representation.

In such a way, the feature representation generated according to the embodiments of the present disclosure can take into account the influence of secondary structure, and the decoder can be used to directly generate the amino acid sequence and secondary structure of the target polypeptide molecule. Thus, the embodiments of the present disclosure can construct polypeptide molecules with expected secondary structures, thereby improving the antibacterial activity of the constructed polypeptide molecules.

Basic principles and several example implementations of the present disclosure are described below with reference to the accompanying drawings.

### Example Device

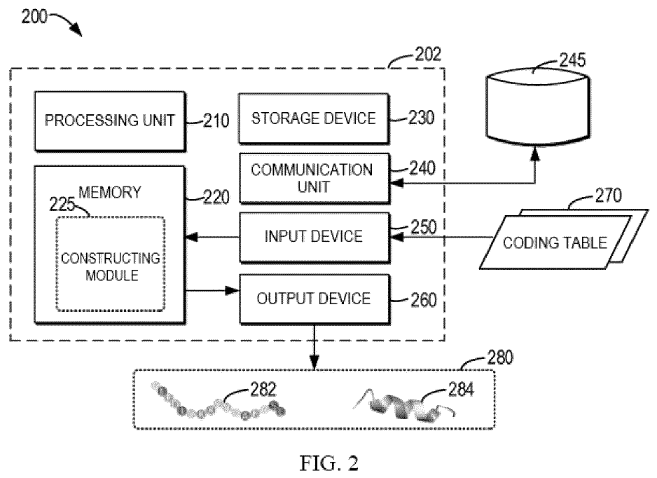

FIG. 2 illustrates a schematic block diagram of an example computing device 200 that may be used to implement embodiments of the present disclosure. It should be understood that the device 200 shown in FIG. 2 is only exemplary and should not constitute any limitation on the functionality and scope of the implementation described in the present disclosure. As shown in FIG. 2, components of device 200 may include, but are not limited to, one or more processors or processing units 210, a memory 220, storage devices 230, one or more communication units 240, one or more input devices 250, and one or more output devices 260.

In some embodiments, the device 200 may be implemented as various user terminals or service terminals. The service terminal may be a server, a large computing device, etc. provided by various service providers. The user terminal may be any type of a mobile terminal, a fixed terminal or a portable terminal, including a mobile phone, a multimedia computer, a multimedia tablet, an Internet node, a communicator, a desktop computer, a laptop computer, a notebook computer, a netbook computer, a tablet computer, a personal communication system (PCS) device, a personal navigation device, a personal digital assistant (PDA), an audio/video player, digital cameras/camcorders, a pointing device, a television receiver, a radio broadcast receiver, a e-book device, a gaming device, or any combinations thereof, including accessories and peripherals of these devices or any combination thereof. It is also contemplated that device 200 can support any type of interface for the user (such as “wearable” circuit, etc.).

The processing unit 220 may be a real or virtual processor and be capable of performing various processes according to a program stored in the memory 220. In a multi-processor system, multiple processing units execute computer-executable instructions in parallel to improve the parallel processing capability of the device 200. The processing unit 220 may also be referred to as a central processing unit (CPU), a microprocessor, a controller, or a microcontroller.

The device 200 typically includes a plurality of computer storage medium. Such medium may be any available medium that is accessible to the device 200, including, but not limited to, a volatile and a nonvolatile medium, a removable and a non-removable medium. Memory 220 may be a volatile memory (e.g., registers, caches, a random access memory (RAM)), a nonvolatile memory (e.g., a read only memory (ROM), an electrically erasable programmable read only memory (EEPROM), a flash memory) or some combination thereof. The Memory 220 may include one or more design modules 225 configured to perform the functions of various implementations described herein. The design module 225 may be accessed and executed by the processing unit 210 to implement corresponding functions. The Storage device 230 may be a removable or a non-removable medium, and may include a machine-readable medium that can be used to store information and/or data and that can be accessed within the device 200.

The functionalities of the components of the device 200 may be implemented with a single computing cluster or multiple computing machines capable of communicating over a communication connection. Thus, the device 200 may operate in a networked environment using logical connections to one or more other servers, personal computers (PCs), or another general network nodes. The device 200 may also communicate with one or more external devices (not shown), such as a database 245, other storage devices, servers, display devices, etc., and with one or more devices that allow the user to communicate with the device 200, or with any device (e.g., a network card, a modem, etc.) that enables the device 200 to communicate with one or more other computing devices, through the communication unit 240 as needed. Such communication may be performed via an input/output (I/O) interface (not shown).

The input device 250 may be one or more various input devices, such as a mouse, a keyboard, a trackball, a voice input device, a camera, etc. The output device 260 may be one or more output devices, such as a display, speakers, a printer, etc.

In some embodiments, as shown in FIG. 2, the device 200 may obtain a set of coding tables 270, which may include, for example, a plurality of trained discrete coded representations. As an example, the device 200 may receive the set of coding tables 270, such as through the input device 250. Alternatively, the device 200 may also read the set of coding tables 270 from the storage device 230 or the database 245. Alternatively, the device 200 may also receive the set of coding tables 270 from other devices through the communication unit 240.

In some embodiments, the constructing module 225 may construct a polypeptide molecule according to the set of coding tables 270. Specifically, the constructing module 225 may determine structural information 280 of the polypeptide molecule, which may include a target amino acid sequence 282 and a target secondary structure 284 of the polypeptide molecule. The process of constructing polypeptide molecules will be described in detail below.

### Training Generation Model

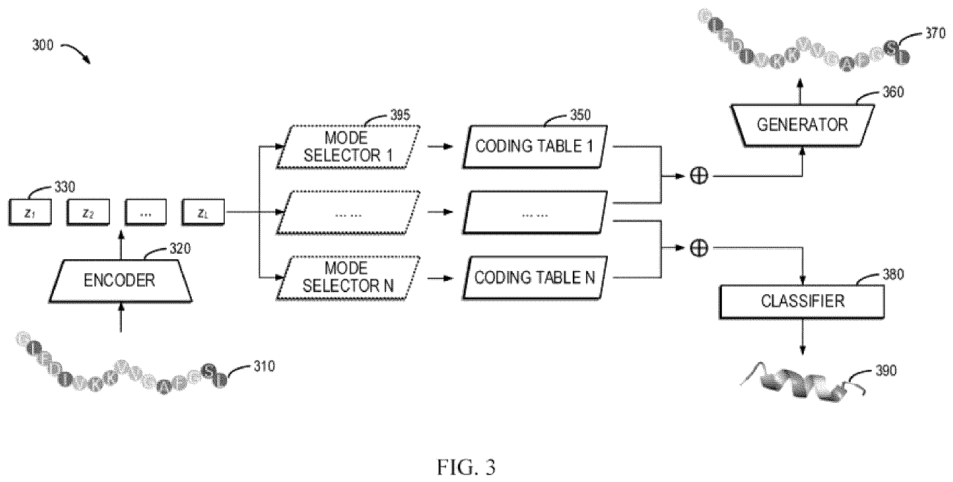

In some embodiments, the constructing module 225 may utilize the generation model to construct the target polypeptide molecule and determine the target amino acid sequence 282 and the target secondary structure 284 of the target polypeptide molecule. In some embodiments, the generation model may be a VQ-VAE model, for example. An example process of training the generation model 300 will be described below with reference to FIG. 3.

As shown in FIG. 3, the generation model 300 may include an encoder 320, a set of coding tables 350, a generator 360, and a classifier 380. In some embodiments, as will be described in detail below, the generation model 300 may also include a set of mode selectors 395.

In some embodiments, the encoder 320 may obtain a set of amino acid sequences 310 of training polypeptide molecules, and then determine a set of amino acid feature representations 330 corresponding to a set of amino acids in the amino acid sequence 310.

Exemplarily, the amino acid sequence 310 of the training polypeptide molecule may be expressed as x={α₁, α₂, . . . , α_{L}} where the α belongs to 20 universal amino acids, and L represents the length of the amino acid sequence 310. The set of amino acid feature representations 330 generated by the encoder 320 may be represented as z=z_{1:L}.

In some embodiments, the generation model 300 may find the discrete coded representation corresponding to each amino acid feature representation 320 by vector quantization. As an example, the generation model 300 may utilize a nearest neighbor search algorithm to search the coding table 350 (for example, it may be expressed as B∈ [symbol] ^{K×d}, where K represents the size of the coding table, d represents the dimension of the entry e in the coding table) for the coding table entry corresponding to the amino acid feature representation 330 (for example, it can be expressed as zₑ(aᵢ)∈ [symbol] ^{d}) generated by the encoder 320, which is also called a discrete coded representation (for example, it can be represented as z_{q}={z_{q}(a₁), . . . , z_{q}(a_{L})}). Therefore, the process may be expressed as:

$${z}_{q} ( {a}_{i} ) = {e}_{k} , k = arg {min}_{j ∈ K} {‖ {z}_{e} ( {a}_{i} ) - {e}_{j} ‖}_{2} \qquad ( 1 )$$

In some embodiments, the feature representations determined by vector quantization by the generation model 300 may be provided to a generator 360 (also referred to as a second decoder) for generating the reconstructed amino acid sequence 370.

In some embodiments, the loss function associated with generating the reconstructed amino acid sequence 370 may be expressed as:

$${L}_{r} = log p ( {a}_{i} \vert {z}_{q} ( {a}_{i} ) ) + {‖ sg [ {z}_{e} ( {a}_{i} ) ] - {z}_{q} ( {a}_{i} ) ‖}_{2}^{2} + β {‖ [ {z}_{e} ( {a}_{i} ) ] - sg [ {z}_{q} ( {a}_{i} ) ] ‖}_{2}^{2} \qquad ( 2 )$$

Among them, sg(·), represents the gradient stopping operator, β represents the weight coefficient; the log p(aᵢ|z_{q}(aᵢ)) portion is designed to make the reconstructed amino acid sequence 370 close to the amino acid sequence 310 of the training polypeptide molecule, that is, related to the processing process of the generator 360;

$${‖ sg [ {z}_{e} ( {a}_{i} ) ] - {z}_{q} ( {a}_{i} ) ‖}_{2}^{2} + β {‖ [ {z}_{e} ( {a}_{i} ) ] - sg [ {z}_{q} ( {a}_{i} ) ] ‖}_{2}^{2}$$

portion represents the difference between the feature representation output by the encoder and the feature representation searched by the coding table, which is intended to make the feature representation output by the encoder close to the feature representation searched by the coding table, that is, it is related to the search process of a set of coding tables 350.

In some embodiments, the secondary structure of the training polypeptide molecule may also be considered in the process of training the generation model 300. For example, the secondary structure of the training polypeptide molecule may be expressed as y={y₁, y₂, . . . , y_{L}}, yᵢ∈{H, B, E, G, I, T, S, -}, where “H” (α-helix), “B” (β-bridge), “E” (folded), “G” (helix-3), “I” (helix-5), “T” (corner), “S” (curl) and “-” (unknown type) respectively represent different secondary structure types.

In some embodiments, the generation model 300 may be trained according to the secondary structure of the training polypeptide molecule. Specifically, the encoder 320 and vector quantization may be utilized to determine the input features

$${z}_{q}^{′} ( {a}_{i} )$$

to the classifier 380 (also referred to as the first decoder). Further, the loss function related to predicting secondary structure may be expressed as:

$${L}_{a} = log p ( {y}_{i} \vert {z}_{q}^{′} ( {a}_{i} ) ) + {‖ sg [ {z}_{e} ( {a}_{i} ) ] - {z}_{q}^{′} ( {a}_{i} ) ‖}_{2}^{2} + β {‖ [ {z}_{e} ( {a}_{i} ) ] - sg [ {z}_{q}^{′} ( {a}_{i} ) ] ‖}_{2}^{2} \qquad ( 3 )$$

Similarly, the

$$log p ( {y}_{i} \vert {z}_{q}^{′} ( {a}_{i} ) )$$

portion is intended to make the predicted secondary structure determined by the classifier 380 close to the secondary structure of the training polypeptide molecule, that is, related the to processing the classifier 380; of

$${‖ sg [ {z}_{e} ( {a}_{i} ) ] - {z}_{q}^{′} ( {a}_{i} ) ‖}_{2}^{2} + β {‖ [ {z}_{e} ( {a}_{i} ) ] - sg [ {z}_{q}^{′} ( {a}_{i} ) ] ‖}_{2}^{2}$$

portion represents the difference between the feature representation output by the encoder and the feature representation searched by the coding table. It is intended to make the feature representation output by the encoder close to the feature representation searched by the coding table, that is, related to the search process of the set of coding tables 350.

In some embodiments, different input features may also be constructed for the generator 360 and classifier 380. As shown in FIG. 3, the generation model 300 may also include a set of mode selectors 395, which may be configured to extract modes with different scales from the set of amino acid feature representations 330 (also referred to as combined feature representations).

The sequence composed of the set of amino acid feature representations 330 may be understood as a mode with a scale of 0; a mode with a scale of 1 may be understood as a mode of the sequence corresponding to each amino acid; a mode with a scale of n may be understood as a mode of this sequence corresponding to all the subsequence of length n.

Accordingly, the mode selector 395 may determine one or more amino acid sub-sequences that match the corresponding length based on a set of amino acids in the amino acid sequence 310, and further determine the corresponding combined feature representation based on the one or more amino acid sub-sequences. The modes with different scales extracted by a set of mode selectors 395 may be represented as:

$${z}_{ε}^{( n )} ( {a}_{i} ) = {F}^{( n )} ( {h}_{i} ) \qquad ( 4 )$$

Wherein, F⁽ⁿ⁾ represents the processing process of a set of selectors 350, and hᵢ represents a set of amino acid feature representations 330 output by the encoder 320.

Further, the generation model 300 may utilize a set of coding tables 360 to update multiple combined feature representations

$${z}_{e}^{( n )} ( {a}_{i} )$$

generated by a set of mode selectors 395 to obtain multiple updated combined feature representations

$${z}_{q}^{( n )} ( {a}_{i} ) ,$$

also known as target discrete coded representations.

In some embodiments, the generation model may generate input feature representations to generator 360 based on multiple updated combined feature representations. In some embodiments, the generation model 300 may select a set of combined feature representations (also referred to as a set of discrete coded representations) from a plurality of updated combined feature representations to construct the input feature representations to the generator 360.

As an example, the input feature representations to the generator 360 may be expressed as:

$${z}_{{q}_{r}} ( {a}_{i} ) = ‖ n ∈ {N}_{r} {z}_{q}^{( n )} ( {a}_{i} ) \qquad ( 5 )$$

where, Nᵣ represents the set of coding tables selected to construct the input feature representations to the generator, and ∥ represents the cascade operation.

Correspondingly, based on this approach, the representation of the loss function (2) may be updated as:

$${L}_{r} = log p ( {a}_{i} \vert {z}_{{q}_{r}} ( {a}_{i} ) ) + {\sum }_{n ∈ {N}_{r}} ( {‖ sg [ {z}_{e}^{( n )} ( {a}_{i} ) ] - {z}_{q}^{( n )} ( {a}_{i} ) ‖}_{2}^{2} + β {‖ sg [ {z}_{e}^{( n )} ( {a}_{i} ) ] - sg [ {z}_{q}^{( n )} ( {a}_{i} ) ] ‖}_{2}^{2} ) \qquad ( 6 )$$

Based on a similar way, the representation of the loss function (3) may further be updated to obtain Lₛ. Further, the total loss function used to train the generation model 300 may be expressed as:

$$L = {L}_{r} + γ {L}_{s} \qquad ( 7 )$$

wherein, γ represents the weight coefficient. Thus, the embodiments of the present disclosure may take the influence of secondary structure into account in the process of training the generation model.

In some embodiments, the known AMP polypeptide molecules may be used to train the generation model 300. Considering the limitations of known AMP polypeptide molecule data sets, large protein data sets may also be used to pre-train the sequence construction task, and polypeptide data sets including protein information may be used to pre-train the secondary structure classification task. Furthermore, the AMP polypeptide molecule data set may be used to tune the generated model.

It should be understood that any suitable VQ-VAE model training method (e.g., using an exponential moving average EMA to update the coding table) may be utilized to train the generation model based on the loss function discussed above.

### Constructing Polypeptide Molecules

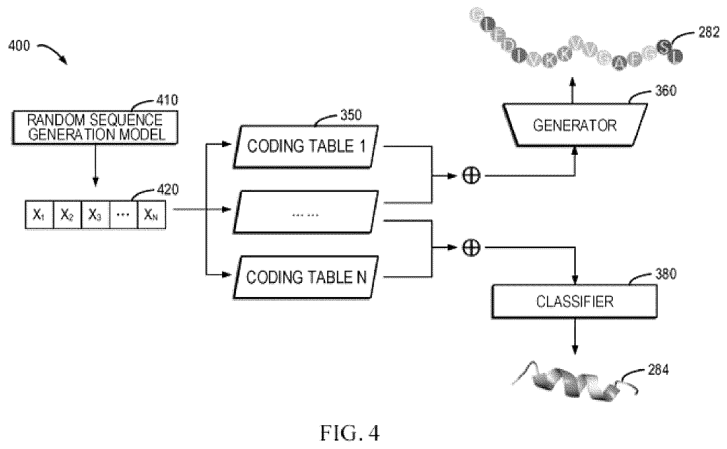

After completing the training of the generation model 300, the constructing module 225 may further utilize a set of coding tables 350 in the generation model 300 to construct polypeptide molecules. It will be understood that the construction device (e.g., the device 200) used to construct the polypeptide molecule may be a different or the same device as the training device used to train the generation model 300. An example process for constructing a polypeptide molecule will be described below with reference to FIG. 4.

As shown in FIG. 4, the construction device may construct a feature representation to the generator 360 and a feature representation to the classifier 380 based on a set of coding tables 350 in the generation model 300.

In some embodiments, the construction device may determine the index sequence 420. The index sequence may, for example, include a plurality of index values X₁-X_{N}, wherein each index value may indicate a selected discrete coded representation in the corresponding coding table.

Further, the construction device may construct a feature representation to the classifier 380 (also referred to as a first feature representation) and a feature representation to the generator 360 (also referred to as a second feature representation) based on a plurality of target discrete coded representations selected in a set of coding tables 350. It should be understood that the feature representation to the generator 360 and the feature representation to the classifier 380 may be constructed using the construction process discussed with reference to equation (5).

Specifically, the construction device may construct the first feature representation based on a first set of discrete coded representations among the plurality of target discrete coded representations, and construct the second feature representation based on a second set of discrete coded representations among the plurality of target discrete coded representations.

In some embodiments, the first set of discrete coded representations may be different from the second set of discrete coded representations. For example, the first set of discrete coded representations may correspond to the 1^{st} to m^{th} coding tables, and the second set of discrete coded representations may correspond to the (m+1)^{th} to N^{th} coding tables.

In some embodiments, the first set of discrete coded representations may at least partially overlap with the second set of discrete coded representations. For example, the first set of discrete coded representations may correspond to the 1^{st} to m^{th} coding tables, and the second set of discrete coded representations may correspond to the m^{th} to N^{th} coding tables. Both sets of discrete coded representations may include the target discrete coded representation selected in the m^{th} coding table.

Further, the construction device may utilize the classifier 380 to generate a target secondary structure 284 of the target polypeptide molecule based on the first feature representation. Correspondingly, the construction device may also utilize the generator 360 to generate the target amino acid sequence 282 of the target polypeptide molecule based on the second feature representation.

Based on this approach, the embodiments of the present disclosure can not only provide the amino acid sequence of the target polypeptide molecule, but also provide the secondary structure of the target polypeptide molecule.

In some embodiments, as shown in FIG. 4, the index sequence 420 may be generated by the construction device with a random sequence generation model 410. In some embodiments, the random sequence generation model is trained on a set of training index sequences for a set of training polypeptide molecules, wherein the set of training index sequences indicates the selected discrete coded representations in the plurality of coding tables.

After completing the training of the random sequence generation model 410, the construction device may, for example, use the random sequence generation model 410 to generate the index sequence 420 based on the initial input or randomly generate the index sequence 420.

In some embodiments, the construction device may firstly determine whether the generated target secondary structure 284 satisfies structural constraints. In some embodiments, the structural constraints may include, for example, a constraint on the proportion of irregular curls in the secondary structure. For example, the proportion of irregular curls needs to be less than 30%. Alternatively, the structural constrains may further include for example the constrain on a length of the alpha helix in the secondary structure. For example, the length of the alpha helix needs to be greater than 4. Through such structural constraints, the antibacterial activity of the generated target polypeptide molecules can be guaranteed.

Further, if it is determined that the target secondary structure satisfies the structural constraints, the construction device further utilizes the second decoder to determine a target amino acid sequence 282 of the target polypeptide molecule according to the second feature representation.

Conversely, if it is determined that the target secondary structure satisfies the structural constraints, the construction device may discard the index sequence. Additionally, the construction device may construct a new first feature representation and a new second feature representation based on a plurality of discrete coded representations of a set of coding tables. For example, the construction device may utilize the random sequence generation model 410 to generate new random sequences.

In some embodiments, the construction device can also generate multiple index sequences at once and discard the index sequences in which the predicted secondary structure does not satisfy the structural constraints.

Based on the process of constructing polypeptide molecules discussed above, the embodiments of the present disclosure can enable input features to fully consider the effect of secondary structure, thereby enabling the construction of polypeptide molecules (for example, antimicrobial peptides) with better antibacterial activity.

### Example Process

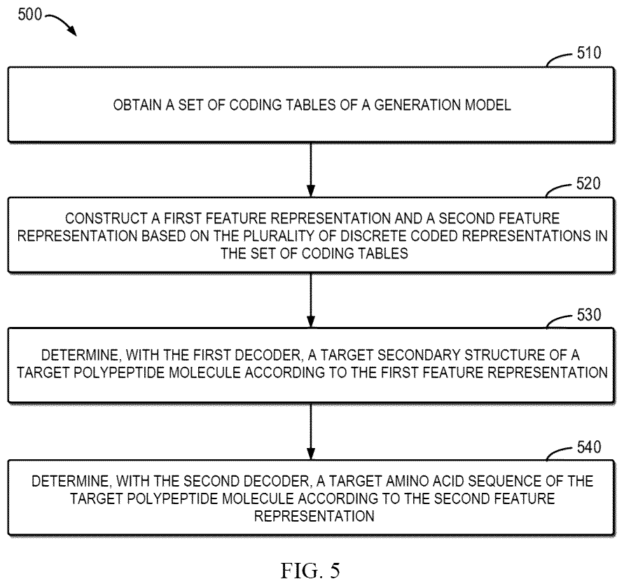

FIG. 5 shows a flow diagram of a method 600 for constructing polypeptide molecules in accordance with some implementations of the present disclosure. The method 500 may be implemented by a computing device 200, for example, the method 500 may be implemented at the constructing block 225 in the memory 220 of the computing device 200.

As shown in FIG. 5, at block 510, the computing device 200 obtains a set of coding tables of a generation model, the set of coding tables comprising a plurality of discrete coded representations, the generation model comprising a first decoder and a second decoder, the set of coding tables being used for constructing a first input to the first decoder and a second input to the second decoder, the first decoder being used for determining a secondary structure of a polypeptide molecule based on the first input, and the second decoder being used for determining an amino acid sequence of the polypeptide molecule based on the second input.

At block 520, the computing device 200 constructs a first feature representation and a second feature representation based on the plurality of discrete coded representations in the set of coding tables.

At block 530, the computing device 200 determines, with the first decoder, a target secondary structure of a target polypeptide molecule according to the first feature representation.

At block 540, the computing device 200 determines, with the second decoder, a target amino acid sequence of the target polypeptide molecule according to the second feature representation.

It should be understood that FIG. 5 is not intended to limit the execution order of the steps corresponding to each block. For example, the steps of blocks 530 and 540 may be performed in parallel, block 530 may be performed prior to block 540, or block 540 may be performed prior to block 530.

In some embodiments, the set of coding tables may comprise a plurality of coding tables, each of which includes a set of discrete coded representations.

In some embodiments, constructing the first feature representation and the second feature representation comprises: determining an index sequence which includes a plurality of index values, each index value indicating a selected target discrete coded representation in the corresponding coding table, and constructing the first feature representation and the second feature representation based on a plurality of selected target discrete coded representations of a plurality of coding tables.

In some embodiments, constructing the first feature representation and the second feature representation based on a plurality of selected target discrete coded representations of a plurality of coding tables comprises: constructing the first feature representation based on a first set of discrete coded representations of the plurality of target discrete coded representations, and constructing the second feature representation based on a second set of discrete coded representations of the plurality of target discrete coded representations, wherein the first set of discrete coded representations is different from the second set of discrete coded representations.

In some embodiments, determining an index sequence comprises: determining the index sequence with a random sequence generation model that is trained for a set of training index sequences of a set of training polypeptide molecules, the set of training index sequences indicating the selected discrete coded representations in the plurality of coding tables.

In some embodiments, determining, with the second decoder, a target amino acid sequence of the target polypeptide molecule according to the second feature representation comprises: determining whether the target secondary structure satisfies structural constraints, the structural constraints including at least one of the following: a constraint on the proportion of irregular curls in the secondary structure, or on the length of the alpha helix in the secondary structure, and determine, with the second decoder, a target amino acid sequence of the target polypeptide molecule according to the second feature representation, in response to determining that the target secondary structure satisfies the structural constraints.

In some embodiments, the method 600 further comprises: constructing a new first feature representation and a new second feature representation based on the plurality of discrete coded representations in the set of coding tables, in response to determining that the target secondary structure satisfies the structural constraints.

In some embodiments, a set of coding tables includes a plurality of coding tables, and the generation model is trained based on the following process: determining, with an encoder of the generation model, a set of amino acid feature representations corresponding to a set of amino acids of the training polypeptide molecules; generating a plurality of combined feature representations corresponding to a plurality of amino acid sequence lengths according to the set of amino acid feature representations; updating the plurality of combined feature representations with the plurality of coding table corresponding to the plurality of amino acid sequence lengths; and determining a loss function for training the generation model based on the plurality of updated combined amino acid feature representations.

In some embodiments, generating a plurality of combined feature representations corresponding to a plurality of amino acid sequence lengths according to the set of amino acid feature representations comprises: for a first length of the plurality of amino acid sequence lengths, determining a set of amino acid sub-sequences matching the first length based on the set of amino acids; and determining combined feature representations corresponding to the set of amino acid sub-sequences with a set of amino acid feature representations.

In some embodiments, the loss function includes a first portion associated with the first decoder, a second portion associated with the second decoder, and a third portion associated with the updating with the plurality of coding tables.

In some embodiments, a first trained input to the first decoder is determined by updating a first initial input with the plurality of coding tables, and a second trained input to the second decoder is determined by updating a second initial input with the plurality of coding tables, and the third portion is determined based on a first difference between the first initial input and the first training input and a second difference between the second initial input and the second training input.

The functions described above herein may be performed, at least in part, by one or more hardware logic components. For example, and without limitation, exemplary types of hardware logic components that may be used include: Field Programmable Gate Arrays (FPGAs), Application Specific Integrated Circuits (ASICs), Application Specific Standard Products (ASSPs), Systems on Chips system (SOCs), Load Programmable Logic Devices (CPLDs) and the like.

Program code for implementing the methods of the present disclosure may be written in any combination of one or more programming languages. These program codes may be provided to a processor or controller of a general-purpose computer, special-purpose computer, or other programmable data processing device, such that the program codes, when executed by the processor or controller, cause the functions/operations specified in the flowcharts and/or block diagrams are implemented. The program code may execute entirely on the machine, partly on the machine, as a stand-alone software package partly on the machine and partly on a remote machine or entirely on the remote machine or server.

In the context of this disclosure, a machine-readable medium may be a tangible medium that may contain or store a program for use by or in connection with an instruction execution system, apparatus, or device. The machine-readable medium may be a machine-readable signal medium or a machine-readable storage medium. The machine-readable medium may include, but are not limited to, electronic, magnetic, optical, electromagnetic, infrared, or semiconductor systems, apparatus or devices, or any suitable combination of the foregoing. More specific examples of machine-readable storage medium would include one or more wires based electrical connection, portable computer disk, hard disk, random access memory (RAM), read only memory (ROM), erasable programmable read only memory (EPROM or flash memory), optical fiber, portable compact disk read-only memory (CD-ROM), optical storage device, magnetic storage device, or any suitable combination of the above.

Furthermore, although operations are depicted in a specific order, this should be understood to require that such operations be performed in the specific order shown or in sequential order, or that all illustrated operations should be performed to achieve desirable results. Under certain circumstances, multitasking and parallel processing may be advantageous. Likewise, although several specific implementation details are included in the above discussion, these should not be construed as limiting the scope of the present disclosure. Certain features that are described in the context of separate implementations can also be implemented in combination in a single implementation. Conversely, various features that are described in the context of a single implementation can also be implemented in multiple implementations separately or in any suitable sub-combination.

Although the present subject matter has been described in language specific to structural features and/or methodological logical acts, it should be understood that the subject matter defined in the accompanying claims is not necessarily limited to the specific features or acts described above. Rather, the specific features and actions described above are merely example forms of implementing claims.

1. A method for constructing a polypeptide molecule, comprising:
obtaining a set of coding tables of a generation model, the set of coding tables comprising a plurality of discrete encoded representations, the generation model comprising a first decoder and a second decoder, the set of coding tables being used for constructing a first input to the first decoder and a second input to the second decoder, the first decoder being used for determining a secondary structure of a polypeptide molecule based on the first input, the second decoder being used for determining an amino acid sequence of the polypeptide molecule based on the second input;
constructing a first feature representation and a second feature representation based on the plurality of discrete encoded representations in the set of coding tables;
determining, with the first decoder, a target secondary structure of a target polypeptide molecule according to the first feature representation; and
determining, with the second decoder, a target amino acid sequence of the target polypeptide molecule according to the second feature representation.

2. The method of claim 1, wherein the set of coding tables comprises a plurality of coding tables, each coding table comprising a set of discrete encoded representations.

3. The method of claim 2, wherein constructing the first feature representation and the second feature representation comprises:
determining an index sequence, the index sequence comprising a plurality of index values, each index value indicating a selected target discrete encoded representation in a corresponding coding table; and
constructing the first feature representation and the second feature representation based on a plurality of selected target discrete encoded representations in the plurality of coding tables.

4. The method of claim 3, wherein constructing the first feature representation and the second feature representation based on a plurality of selected target discrete encoded representations in the plurality of coding tables comprises:
constructing the first feature representation based on a first set of discrete encoded representations of the plurality of target discrete encoded representations; and
constructing the second feature representation based on a second set of discrete encoded representations of the plurality of target discrete encoded representations, the first set of discrete coded representations being different from the second set of discrete coded representations.

5. The method of claim 3, wherein determining an index sequence comprises:
determining the index sequence with a random sequence generation model, the random sequence generation model being trained for a set of training index sequences of a set of training polypeptide molecules, the set of training index sequences indicating a selected discrete encoded representation in the plurality of coding tables.

6. The method of claim 1, wherein determining, with the second decoder, a target amino acid sequence of the target polypeptide molecule according to the second feature representation comprises:
determining whether the target secondary structure satisfies structural constraint, the structural constraint comprising at least one of the following: a constraint on a proportion of an irregular curl in the secondary structure, or a constraint on a length of an alpha helix in the secondary structure; and
in response to determining that the target secondary structure satisfies the structural constraint, determining, with the second decoder, a target amino acid sequence of the target polypeptide molecule according to the second feature representation.

7. The method of claim 6, further comprising:
in response to determining that the target secondary structure satisfies the structural constraint, constructing a new first feature representation and a new second feature representation based on the plurality of discrete encoded representations in the set of coding tables.

8. The method of claim 1, wherein the set of coding tables comprises a plurality of coding tables, and the generation model is trained based on the following process:
determining, with an encoder of the generation model, a set of amino acid feature representations corresponding to a set of amino acids in a training polypeptide molecule;
generating a plurality of combined feature representations corresponding to a plurality of amino acid sequence lengths according to the set of amino acid feature representations;
updating the plurality of combined feature representations with the plurality of coding tables corresponding to the plurality of amino acid sequence lengths; and
determining a loss function for training the generation model based on the plurality of updated combined amino acid feature representations.

9. The method of claim 8, wherein generating a plurality of combined feature representations corresponding to a plurality of amino acid sequence lengths according to the set of amino acid feature representations comprises:
for a first length of the plurality of amino acid sequence lengths,
determining a set of amino acid sub-sequences matching the first length based on the set of amino acids; and
determining a combined feature representation corresponding to the set of amino acid sub-sequences using a set of amino acid feature representations.

10. The method of claim 8, wherein the loss function comprises a first portion associated with the first decoder, a second portion associated with the second decoder, and a third portion associated with the updating with the plurality of coding tables.

11. The method of claim 10, wherein a first training input to the first decoder is determined by updating a first initial input with the plurality of coding tables, and a second training input to the second decoder is determined by updating a second initial input with the plurality of coding tables, and the third portion is determined based on a first difference between the first initial input and the first training input and a second difference between the second initial input and the second training input.

12. An electronic device, comprising:
a memory and a processor;
wherein the memory is used to store one or more computer instructions, wherein the one or more computer instructions are executed by the processor to implement a method for constructing a polypeptide molecule comprising:
obtaining a set of coding tables of a generation model, the set of coding tables comprising a plurality of discrete encoded representations, the generation model comprising a first decoder and a second decoder, the set of coding tables being used for constructing a first input to the first decoder and a second input to the second decoder, the first decoder being used for determining a secondary structure of a polypeptide molecule based on the first input, the second decoder being used for determining an amino acid sequence of the polypeptide molecule based on the second input;
constructing a first feature representation and a second feature representation based on the plurality of discrete encoded representations in the set of coding tables;
determining, with the first decoder, a target secondary structure of a target polypeptide molecule according to the first feature representation; and
determining, with the second decoder, a target amino acid sequence of the target polypeptide molecule according to the second feature representation.

13. A non-transitory computer-readable storage medium having one or more computer instructions stored thereon, wherein the one or more computer instructions are executed by a processor to implement a method for constructing a polypeptide molecule comprising:
obtaining a set of coding tables of a generation model, the set of coding tables comprising a plurality of discrete encoded representations, the generation model comprising a first decoder and a second decoder, the set of coding tables being used for constructing a first input to the first decoder and a second input to the second decoder, the first decoder being used for determining a secondary structure of a polypeptide molecule based on the first input, the second decoder being used for determining an amino acid sequence of the polypeptide molecule based on the second input;
constructing a first feature representation and a second feature representation based on the plurality of discrete encoded representations in the set of coding tables;
determining, with the first decoder, a target secondary structure of a target polypeptide molecule according to the first feature representation; and
determining, with the second decoder, a target amino acid sequence of the target polypeptide molecule according to the second feature representation.

14. (canceled)

15. The electronic device of claim 12, wherein the set of coding tables comprises a plurality of coding tables, each coding table comprising a set of discrete encoded representations.

16. The electronic device of claim 13, wherein constructing the first feature representation and the second feature representation comprises:
determining an index sequence, the index sequence comprising a plurality of index values, each index value indicating a selected target discrete encoded representation in a corresponding coding table; and
constructing the first feature representation and the second feature representation based on a plurality of selected target discrete encoded representations in the plurality of coding tables.

17. The electronic device of claim 14, wherein constructing the first feature representation and the second feature representation based on a plurality of selected target discrete encoded representations in the plurality of coding tables comprises:
constructing the first feature representation based on a first set of discrete encoded representations of the plurality of target discrete encoded representations; and
constructing the second feature representation based on a second set of discrete encoded representations of the plurality of target discrete encoded representations, the first set of discrete coded representations being different from the second set of discrete coded representations.

18. The electronic device of claim 14, wherein determining an index sequence comprises:
determining the index sequence with a random sequence generation model, the random sequence generation model being trained for a set of training index sequences of a set of training polypeptide molecules, the set of training index sequences indicating a selected discrete encoded representation in the plurality of coding tables.

19. The electronic device of claim 12, wherein determining, with the second decoder, a target amino acid sequence of the target polypeptide molecule according to the second feature representation comprises:
determining whether the target secondary structure satisfies structural constraint, the structural constraint comprising at least one of the following: a constraint on a proportion of an irregular curl in the secondary structure, or a constraint on a length of an alpha helix in the secondary structure; and
in response to determining that the target secondary structure satisfies the structural constraint, determining, with the second decoder, a target amino acid sequence of the target polypeptide molecule according to the second feature representation.

20. The electronic device of claim 17, the method further comprising:
in response to determining that the target secondary structure satisfies the structural constraint, constructing a new first feature representation and a new second feature representation based on the plurality of discrete encoded representations in the set of coding tables.

21. The electronic device of claim 12, wherein the set of coding tables comprises a plurality of coding tables, and the generation model is trained based on the following process:
determining, with an encoder of the generation model, a set of amino acid feature representations corresponding to a set of amino acids in a training polypeptide molecule;
generating a plurality of combined feature representations corresponding to a plurality of amino acid sequence lengths according to the set of amino acid feature representations;
updating the plurality of combined feature representations with the plurality of coding tables corresponding to the plurality of amino acid sequence lengths; and
determining a loss function for training the generation model based on the plurality of updated combined amino acid feature representations.
